(19) United States
(12) Patent Application Publication
(10) Pub. No.: US 2007/0167771 A1
(43) Pub. Date: Jul. 19, 2007

(54) ULTRASOUND LOCATION OF ANATOMICAL LANDMARKS

(75) Inventor: Bjorn Olstad, 3960 Stathelle (NO)

Correspondence Address: MCANDREWS HELD & MALLOY, LTD, 500 WEST MADISON STREET, SUITE 3400, CHICAGO, IL, 60661, US

(73) Assignee: G.E. MEDICAL SYSTEMS GLOBAL TECHNOLOGY CO., LLC, Waukesha, WI (US)

(21) Appl. No.: 11/684,507

(22) Filed: Mar. 9, 2007

Related U.S. Application Data
(63) Continuation of application No. 10/248,090, filed on Dec. 17, 2002.

(51) Int. Cl. A61B 8/00

(52) U.S. Cl. 600/437

(57) ABSTRACT

An ultrasound machine is disclosed that includes a method and apparatus for generating an image responsive to moving cardiac structure and for locating anatomical landmarks of the heart by generating received signals in response to ultrasound waves transmitted into and then backscattered from the moving cardiac structure over a time period. A processor is responsive to the received signals to generate a set of analytic parameter values representing movement of the cardiac structure over the time period and analyzes elements of the set of analytic parameter values to automatically extract position information of the anatomical landmarks. A display is arranged to overlay indicia onto the image corresponding to the position information of the anatomical landmarks. The positions of the anatomical landmarks are tracked in real-time.

### RELATED APPLICATIONS

The present application is a continuation of U.S. patent application Ser. No. 10/248,090, entitled “Ultrasound Location Of Anatomical Landmarks,” filed Dec. 17, 2002, which is hereby incorporated by references in its entirety.

### BACKGROUND OF THE INVENTION

Certain embodiments of the present invention relate to an ultrasound machine for locating anatomical landmarks in the heart. More particularly, certain embodiments relate to automatically determining positions of anatomical landmarks of the heart in an image and overlaying indicia on the image that indicate the positions of the anatomical landmarks.

Echocardiography is a branch of the ultrasound field that is currently a mixture of subjective image assessment and extraction of key quantitative parameters. Evaluation of cardiac wall function has been hampered by a lack of well-established parameters that may be used to increase the accuracy and objectivity in the assessment of, for example, coronary artery diseases. Stress echo is such an example. It has been shown that the subjective part of wall motion scoring in stress echo is highly dependent on operator training and experience. It has also been shown that inter-observer variability between echo-centers is unacceptably high due to the subjective nature of the wall motion assessment.

Much technical and clinical research has focused on the problem and has aimed at defining and validating quantitative parameters. Encouraging clinical validation studies have been reported, which indicate a set of new potential parameters that may be used to increase objectivity and accuracy in the diagnosis of, for instance, coronary artery diseases. Many of the new parameters have been difficult or impossible to assess directly by visual inspection of the ultrasound images generated in real-time. The quantification has typically required a post-processing step with tedious, manual analysis to extract the necessary parameters. Determination of the location of anatomical landmarks in the heart is no exception. Time intensive post-processing techniques or complex, computation-intensive real-time techniques are undesirable.

A method in U.S. Pat. No. 5,601,084 to Sheehan et al. describes imaging and three-dimensionally modeling portions of the heart using imaging data. A method in U.S. Pat. No. 6,099,471 to Torp et al. describes calculating and displaying strain velocity in real time. A method in U.S. Pat. No. 5,515,856 to Olstad et al. describes generating anatomical M-mode displays for investigations of living biological structures, such as heart function, during movement of the structure. A method in U.S. Pat. No. 6,019,724 to Gronningsaeter et al. describes generating quasi-realtime feedback for the purpose of guiding procedures by means of ultrasound imaging.

A need exists for a simple, real-time technique for automatic localization, indication, and tracking of anatomical landmarks of the heart, such as the apex and the atrium/ventricle (AV) plane.

### BRIEF SUMMARY OF THE INVENTION

An embodiment of the present invention provides an ultrasound system for imaging a heart, automatically locating anatomical landmarks within the heart, overlaying indicia onto the image of the heart corresponding to the positions of the anatomical landmarks, and tracking the anatomical landmarks.

An apparatus is provided in an ultrasound machine for overlaying indicia onto a displayed image responsive to moving structure within the heart of a subject such that the indicia indicate locations of anatomical landmarks within the heart. In such an environment an apparatus displaying the indicia preferably comprises a front-end arranged to transmit ultrasound waves into a structure and to generate received signals in response to ultrasound waves backscattered from said structure over a time period. A processor is responsive to the received signals to generate a set of analytic parameter values representing movement of the cardiac structure over the time period and analyzes elements of the set of analytic parameter values to automatically extract position information of the anatomical landmarks and track the positions of the landmarks. A display is arranged to overlay indicia corresponding to the position information onto an image of the moving structure to indicate to an operator the position of the tracked anatomical landmarks.

A method is also provided in an ultrasound machine for overlaying indicia onto a displayed image responsive to moving structure within the heart of a subject such that the indicia indicate locations of anatomical landmarks within the heart. In such an environment a method for displaying the indicia preferably comprises transmitting ultrasound waves into a structure and generating received signals in response to ultrasound waves backscattered from said structure over a time period. A set of analytic parameter values is generated in response to the received signals representing movement of the cardiac structure over the time period. Position information of the anatomical landmarks is automatically extracted and the positions of the landmarks are then tracked. Indicia corresponding to the position information are overlaid onto the image of the moving structure to indicate to an operator the position of the tracked anatomical landmarks.

Certain embodiments of the present invention afford a relatively simple approach to automatically locate key anatomical landmarks of the heart, such as the apex and the AV-plane, and track the landmarks with a degree of convenience and accuracy previously unattainable in the prior art.

### BRIEF DESCRIPTION OF THE DRAWINGS

FIG. 1 is a schematic block,diagram of an ultrasound machine made in accordance with an embodiment of the present invention.

FIG. 2 is a flowchart of a method performed by the machine shown in FIG. 1 in accordance with an embodiment of the present invention.

FIG. 3 illustrates an apical cross section of a heart and shows an illustration of an exemplary tissue velocity image of a heart generated by the ultrasound machine in FIG. 1 in accordance with an embodiment of the present invention.

FIG. 4 illustrates an exemplary resultant motion gradient profile derived from analytic parameter values comprising tissue velocity values, and also shows designated anatomical points along a length of a myocardial segment in accordance with an embodiment of the present invention.

FIG. 5 is an exemplary pair of graphs of a tracked velocity parameter profile and a motion parameter profile generated by a longitudinal tracking function executed by the ultrasound machine in FIG. 1 and corresponding to a designated point in a myocardial segment, in accordance with an embodiment of the present invention.

FIG. 6 illustrates several exemplary tissue velocity estimate profiles at discrete points along a color image of a myocardial segment of a heart indicating motion over a designated time period in accordance with an embodiment of the present invention.

FIG. 7 illustrates exemplary indicia overlaid onto an image of the heart, indicating landmarks of the heart in accordance with an embodiment of the present invention.

FIG. 8 illustrates the motion of the indicia shown in FIG. 7 being longitudinally tracked by the ultrasound machine in FIG. 1 in accordance with an embodiment of the present invention.

FIG. 9 illustrates several exemplary velocity profiles, like those shown in FIG. 6, corresponding to discrete points along a myocardial segment of an exemplary color image and indicating peaks in the profiles over a designated time period.

FIG. 10 illustrates the resultant velocity gradient profile derived from the peaks of the exemplary velocity profiles of FIG. 9 in accordance with an embodiment of the present invention.

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

### DETAILED DESCRIPTION OF THE INVENTION

An embodiment of the present invention enables real-time location and tracking of anatomical landmarks of the heart. Moving cardiac structure is monitored to accomplish the function. As used in the specification and claims, structure means non-liquid and non-gas matter, such as cardiac wall tissue. An embodiment of the present invention helps establish improved, real-time visualization and assessment of key anatomical landmarks of the heart such as the apex and the AV-plane. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, and mitral valve inferred values.

FIG. 1 is a schematic block diagram of an embodiment of the present invention comprising an ultrasound machine 5. A transducer 10 is used to transmit ultrasound waves into a subject by converting electrical analog signals to ultrasonic energy and to receive ultrasound waves backscattered from the subject by converting ultrasonic energy to analog electrical signals. A front-end 20 comprising a receiver, transmitter, and beamformer, is used to create the necessary transmitted waveforms, beam patterns, receiver filtering techniques, and demodulation schemes that are used for the various imaging modes. Front-end 20 performs the functions by converting digital data to analog data and vice versa. Front-end 20 interfaces at an analog interface 15 to transducer 10 and interfaces over a digital bus 70 to a non-Doppler processor 30 and a Doppler processor 40 and a control processor 50. Digital bus 70 may comprise several digital sub-buses, each sub-bus having its own unique configuration and providing digital data interfaces to various parts of the ultrasound machine 5.

Non-Doppler processor 30 comprises amplitude detection functions and data compression functions used for imaging modes such as B-mode, B M-mode, and harmonic imaging. Doppler processor 40 comprises clutter filtering functions and movement parameter estimation functions used for imaging modes such as tissue velocity imaging (TVI), strain rate imaging (SRI), and color M-mode. The two processors, 30 and 40, accept digital signal data from the front-end 20, process the digital signal data into estimated parameter values, and pass the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 comprises scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and passes the analog display signals to a monitor 90. Monitor 90 accepts the analog display signals from display processor 80 and displays the resultant image to the operator on monitor 90.

A user interface 60 allows user commands to be input by the operator to the ultrasound machine 5 through control processor 50. User interface 60 comprises a keyboard, mouse, switches, knobs, buttons, track ball, and on screen menus.

A timing event source 65 is used to generate a cardiac timing event signal 66 that represents the cardiac waveform of the subject. The timing event signal 66 is input to ultrasound machine 5 through control processor 50.

Control processor 50 is the main, central processor of the ultrasound machine 5 and interfaces to various other parts of the ultrasound machine 5 through digital bus 70. Control processor 50 executes the various data algorithms and functions for the various imaging and diagnostic modes. Digital data and commands may be transmitted and received between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

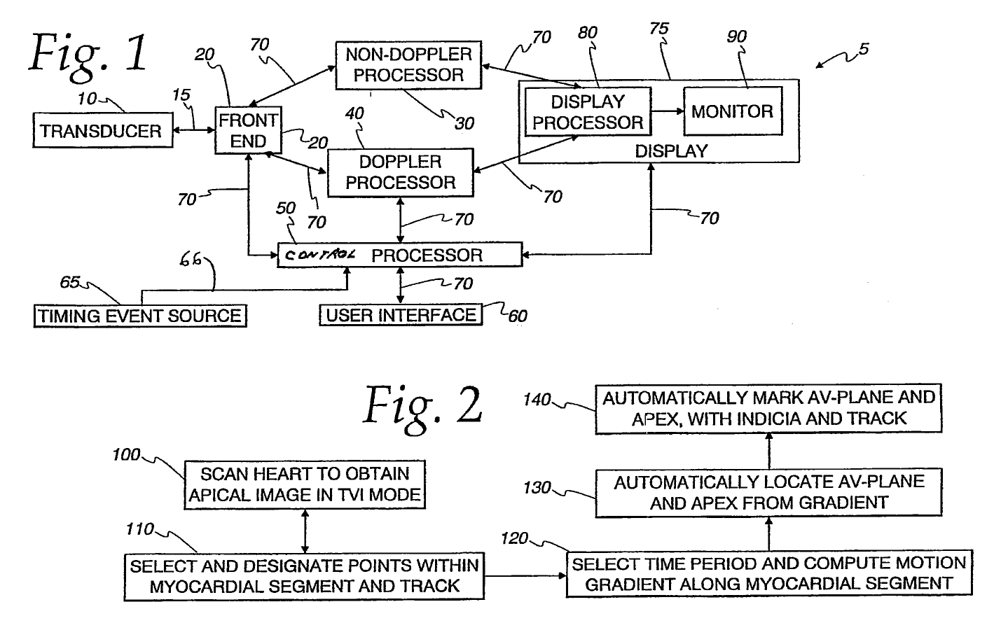

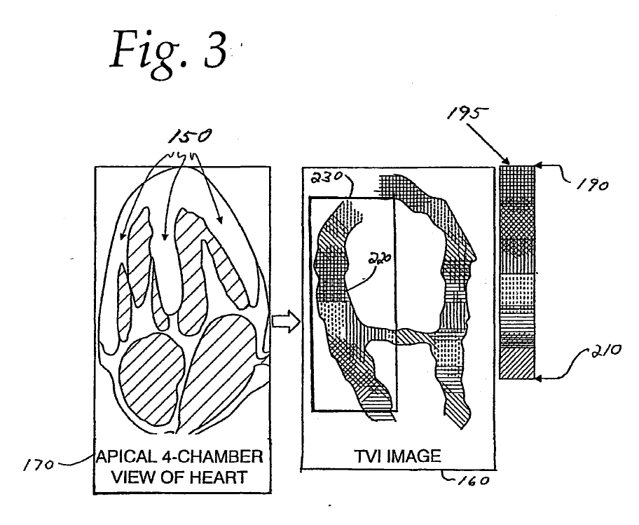

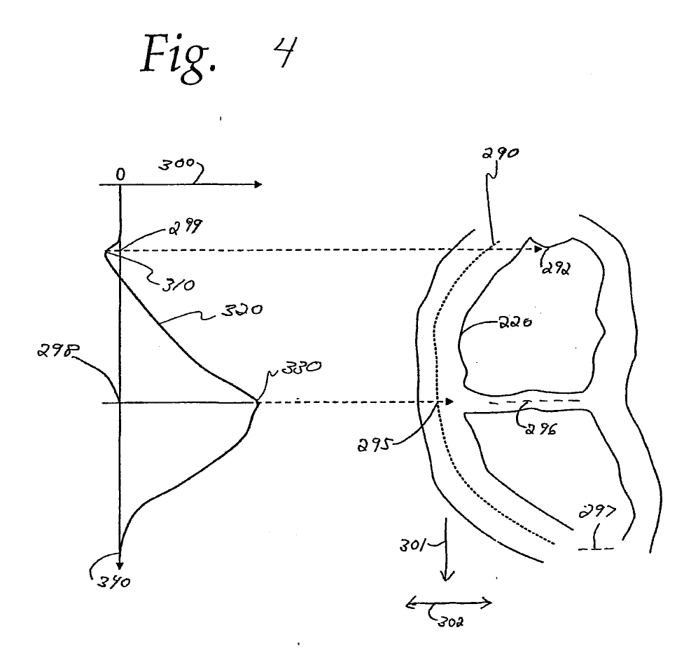

Referring to FIG. 2, according to an embodiment of the present invention, in step 100 an operator uses transducer 10 to transmit ultrasound energy into anatomical structure, such as cardiac tissue 150 (see FIG. 3), of the subject in an imaging mode, such as tissue velocity imaging (TVI) 160, that will yield the desired set of analytic parameter values of the desired anatomical structure (typically a 2-dimensional apical cross section of the heart 170). Ultrasound energy is received into transducer 10 and signals are received into front-end 20 in response to ultrasound waves backscattered from the structure. The resultant analytic parameter values computed by non-Doppler processor 30 and/or Doppler processor 40 typically comprise estimates of at least one of tissue velocity, B-mode tissue intensity , and tissue strain rate.

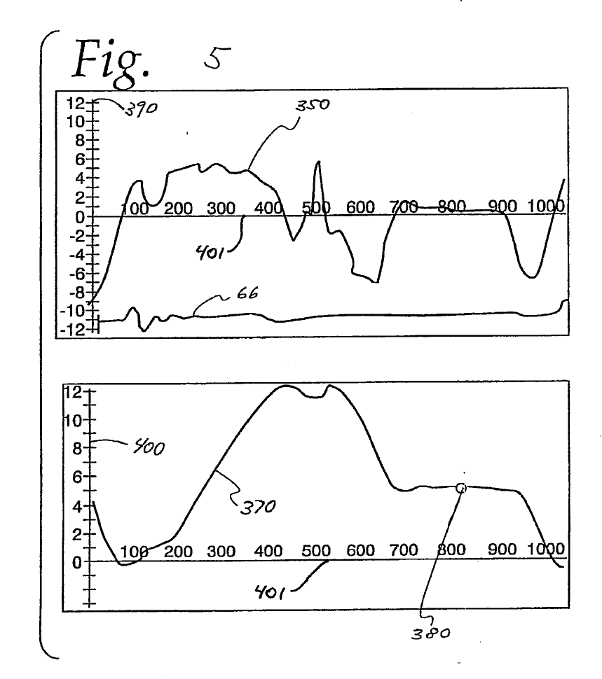

In an embodiment of the present invention, in step 110 of FIG. 2, the operator brings up a region-of-interest (ROI) 230 on monitor 90 through the user interface 60 to designate anatomical points along a myocardial segment 220 of the heart in the color TVI image of imaging mode 160 on monitor 90. The color legend 195 indicates the tissue velocity values within the myocardial segment 220 in the TVI imaging mode 160. The analytic parameter values (e.g. tissue velocity values) corresponding to the desired myocardial segment 220 are automatically separated from the parameter values of cavities and other cardiac structure of the heart by processor 50 using, for example, B-mode tissue intensity in conjunction with a segmentation algorithm in accordance with an embodiment of the present invention. Anatomical points 290 (see FIG. 4) are automatically designated within the myocardial segment 220. Well-known segmentation, thresholding, centroiding, and designation techniques operating on at least one of the set of analytic parameter values are used to establish the designated points 290 in accordance with an embodiment of the present invention.

Such a designation of a myocardial segment 220 will force the automatic extraction and subsequent processing of the set of analytic parameter values and the display of the resultant anatomical landmark positions of the heart. As an alternative embodiment of the present invention, instead of the operator defining a ROI 230 around the myocardial segment 220, the entire image of the TVI imaging mode 160 may be automatically analyzed by host processor 50 to isolate a myocardial segment or multiple segments using automatic segmentation, thresholding, centroiding, and designation techniques in accordance with an embodiment of the present invention.

Once the anatomical points 290 within the desired myocardial segment 220 are designated, real-time tracking of each of the designated points is performed in accordance with an embodiment of the present invention. The set of analytic parameter values corresponding to the designated anatomical points 290 are sent from non-Doppler processor 30 and/or Doppler processor 40 to control processor 50, where a tracking function is applied to at least a subset of the analytic parameter values. FIG. 5 illustrates certain profiles 350 and 370 created by the tracking function in accordance with an embodiment of the present invention. Point 295 (see FIG. 4) is an example of an anatomical point to be tracked.

As an introduction to the tracking function, in accordance with an embodiment of the present invention, a tracked velocity parameter profile 350 (V₁, V₂, . . . , Vₙ) (FIG. 5) for a given sampled anatomical point (e.g. 295) in the myocardium 220, is created by converting a set of estimated tissue velocity values into a motion parameter profile 370 in time by control processor 50. Generation of the profile is accomplished by computing the series of time integrals (S₁, S₂, . . . , Sₙ) where:
Sᵢ=T*(V₁+V₂+. . . +Vᵢ) [1]
and where T is the time delay between two consecutive velocity estimates (T is typically based on the frame rate of the imaging mode). Sᵢ (motion value, e.g. 380) is then the longitudinal distance in millimeters (from some zero reference location 375) that a sample of tissue in the myocardium 295 has moved at time segment Tᵢ, thus allowing the isolated tissue sample to be tracked in a longitudinal direction 301 (along the ultrasound beam) by control processor 50. The tracking function estimates the new spatial location of the anatomical tissue sample after every time segment Tᵢ and extracts velocity estimates at the new spatial locations. The tracking is done for all of the designated anatomical points 290 along the myocardial segment 220.

The upper part of FIG. 5 shows a resultant tracked velocity parameter profile 350 of a designated anatomical point (e.g. 295) in the image as a function of time for a complete cardiac cycle. The velocity scale 390 shows the change in velocity over a time axis 401 in, for example, units of cm/sec. The lower part of FIG. 5 shows the corresponding resultant longitudinal motion parameter profile 370 (time-integrated velocity profile, S₁, S₂, . . . , Sₙ) of the same designated anatomical point (e.g. 295) in the image. The distance axis 400 shows the change in longitudinal deviation over a time axis 401 in units of, for example, millimeters. Motion 300 in millimeters along the ultrasound beam direction 301 may be accurately tracked with the technique allowing the appropriate velocity parameter profiles to be generated for the corresponding anatomical locations. The tracked velocity parameter profile for each designated anatomical point is stored in the memory of control processor 50 as a sampled array of tissue velocity values. As a result, the stored parameter profile history corresponds to each designated anatomical point, instead of just a spatial location in the image.

Two-dimensional velocity estimation is necessary for accurate tracking when a substantial part of the motion of the structure is in an orthogonal direction 302 to the ultrasound beam direction 301. Tracking may be performed in any combination of longitudinal depth, lateral position, and angular position according to various embodiments of the present invention. Other tracking techniques may be employed as well.

The specifics of the preferred tracking function are now described for a given designated anatomical point within a myocardial segment in accordance with an embodiment of the present invention. The methodology generates, at a minimum, a set of tissue velocity values in step 100 of FIG. 2 so that the motion values Sᵢ may be calculated for tracking. The tissue velocity values are generated by Doppler processor 40 in a well-known manner, such as in the TVI imaging mode.

Processor 50 selects a velocity value Vᵢ for a designated anatomical point in the image from a spatial set of estimated tissue velocity values corresponding to a time Tᵢ where i=1 and is called T₁. Processor 50 computes the motion value Sᵢ for the designated anatomical point (e.g. 295), as
Sᵢ=T*(V₁+V₂+. . . +Vᵢ) [1]
(Note that for i=1, S₁=T*V₁)

Processor 50 then stores Vᵢ in a tracked velocity parameter profile array 350 and Sᵢ is stored in a motion parameter profile array 370 along with the current spatial position (e.g. 298) of the designated anatomical point (e.g. 295). Next, i is incremented by one (corresponding to the next sample time, T seconds later) and the next Vᵢ is selected from the spatial set of velocity values based on the motion parameter Sᵢ previously computed and the previous spatial position of the anatomical location in accordance with an embodiment of the present invention (Sᵢ represents the longitudinal spatial movement in millimeters of the designated anatomical point over time interval Tᵢ=i*T).

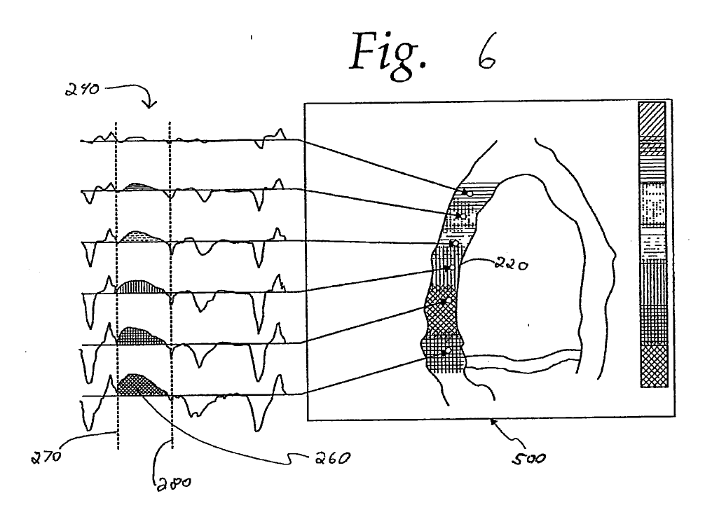

The tracking function then computes the next motion parameter value Sᵢ in the series using Equation [1] in the same manner. The iterative process is followed for continuous tracking of the designated anatomical point. The tracking function is performed simultaneously for each of the designated anatomical points 290 in the myocardial segment. FIG. 5 illustrates the resultant motion parameter profile of a designated anatomical point. The motion parameter profile 370 is a history of the longitudinal movement of the designated anatomical point over time. When estimated tissue velocity values are integrated over time, the resultant motion parameter value (shaded areas 260 of FIG. 6) is a distance moved in units of length such as millimeters (mm).

In step 120 of FIG. 2, the operator selects, through the user interface 60, a desired time period over which to process the estimated analytic parameter values, such as systole, which is a sub-interval of the cardiac cycle in accordance with an embodiment of the present invention. In FIG. 6, the time period is defined by Tₛₜₐᵣₜ 270 and T_{end} 280. The time period is determined from a cardiac timing signal 66 (FIGS. 1 and 6) generated from the timing event source 65 (FIG. 1) and/or from characteristic signatures in estimated analytic parameter values. An example of such a cardiac timing signal is an ECG signal. Those skilled in ultrasound also know how to derive timing events from signals of other sources such as a phonocardiogram signal, a pressure wave signal, a pulse wave signal, or a respiratory signal. Ultrasound modalities such as spectral Doppler or M-modes may also be used to obtain cardiac timing information.

Tₛₜₐᵣₜ 270 is typically selected by the operator as an offset from the R-event in the ECG signal. T_{end} 280 is set such that the time interval covers a selected portion of the cardiac cycle such as systole. It is also possible to select a time period corresponding to the complete cardiac cycle. Other sub-intervals of the cardiac cycle may also be selected in accordance with other embodiments of the present invention.

FIG. 6 graphically illustrates typical sets of estimated parameter profiles 240 of tissue velocity at anatomical points within myocardial tissue 220 in an exemplary color TVI image 500 that may be segmented into desired time periods based on signature characteristics of the sets 240. The time period may be selected automatically or as a combination of manual and automatic methods. For example, the time period could be determined automatically with an algorithm embedded in control processor 50 in accordance with an embodiment of the present invention. The algorithm could use well-known techniques of analyzing the sets of estimated parameter profiles 240, as shown in FIG. 6, looking for key signature characteristics and defining a time period based on the characteristics, or similarly, analyzing the ECG signal (e.g. 66). An automatic function could be implemented to recognize and exclude unwanted events from the selected time period, if desired, as well.

According to an embodiment of the present invention, once the time period is established, the stored, tracked velocity parameter profile array (e.g. 350) for each of the designated anatomical points 290 is integrated over the time period Tₛₜₐᵣₜ 270 to T_{end} 280 by control processor 50 to form motion parameter values over the image depth 340. A time integration function accomplishes the integration in control processor 50 which approximates the true time integral by summing the tracked values as follows:
Sᵢₙₜ=T*(Vₛₜₐᵣₜ+V2+V3+. . . +V_{end}) [2]
where Sᵢₙₜ is the time integrated value (motion parameter value), Vₛₜₐᵣₜ is the value in the tracked velocity parameter profile array corresponding to Tₛₜₐᵣₜ 270 and V_{end} is the value corresponding to T_{end} 280. Each shaded area 260 under the profiles 240 in FIG. 6 represent a motion parameter value calculated by integrating tissue velocity values over the time interval Tₛₜₐᵣₜ 270 to T_{end} 280. The time integration function is performed simultaneously for each of the designated anatomical points 290 in the myocardial segment 220 to form the set of motion parameter values which constitutes a motion gradient profile 320 over the image depth 340, as illustrated in FIG. 4.

Care should be taken by the operator to adjust the Nyquist frequency 190 and 210 of the imaging mode such that aliasing does not occur. With aliasing present in the data, erroneous results may occur. Alternatively, well known automatic aliasing correction techniques may be employed.

In step 130 of FIG. 2, the time integrated velocity parameter value Sᵢₙₜ for each of the designated and tracked anatomical points 290 (the motion gradient profile 370) is used by processor 50 to locate the longitudinal depth position 299 of the apex 292 and the longitudinal depth position 298 of the AV-plane 296 of the heart in the image in accordance with an embodiment of the present invention.

FIG. 4 illustrates an exemplary motion gradient profile 320 corresponding to the designated, tracked anatomical points 290 along the myocardial segment 220 in the image. It may be appreciated how the magnitude 300 of the profile increases (becomes more positive with respect to a zero reference 305) as the sampling location is moved from the apex 292 down toward the AV-plane 296. In particular, the motion values during systole increase from apex 292 down to the AV-plane 296. The motion values attain their peak positive value 330 at or close to the AV-plane 296 and start to decrease as the base of the atrium 297 is approached. Therefore, the peak positive value 330 is used to locate the longitudinal depth 298 of the AV-plane 296.

Also, slightly negative motion values 310 are often found in the apex 292 as a consequence of the myocardial wall thickening in the apex 292. Therefore, the negative peak is used to locate the longitudinal depth 299 of the apex 292. Processor 50 locates the apex 292 and AV-plane 296 by peak-detecting the motion gradient profile 320 over depth 340. In accordance with an embodiment of the present invention, the positive-most peak 330 is searched for and found as the AV-plane 296 location and then the negative peak 310, which is above the AV-plane 296, is searched for and found as the apex 292 location. Even though the AV-plane 296 and apex 292 are clearly shown in the illustration on the right side of FIG. 4, the anatomical locations are often not so apparent in a real displayed image, thus establishing the need for the invention.

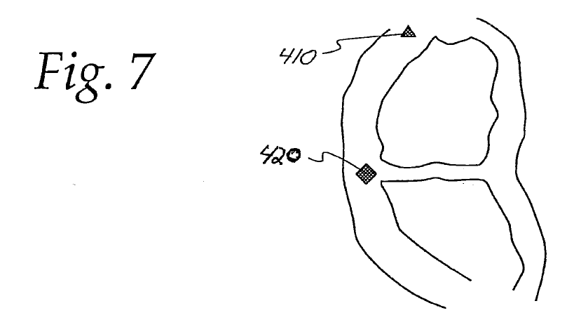

In step 140 of FIG. 2, in accordance with an embodiment of the present invention, discrete anatomical points in the image at the longitudinal depths 298 and 299 of the anatomical landmarks (apex 292 and AV-plane 296) are automatically labeled with indicia 410 and 420 as shown in FIG. 7. The anatomical points are continually tracked, using the techniques described previously, as imaging continues. The positions of the indicia 410 and 420 are continuously updated and displayed to follow the tracked anatomical points corresponding to the anatomical landmarks.

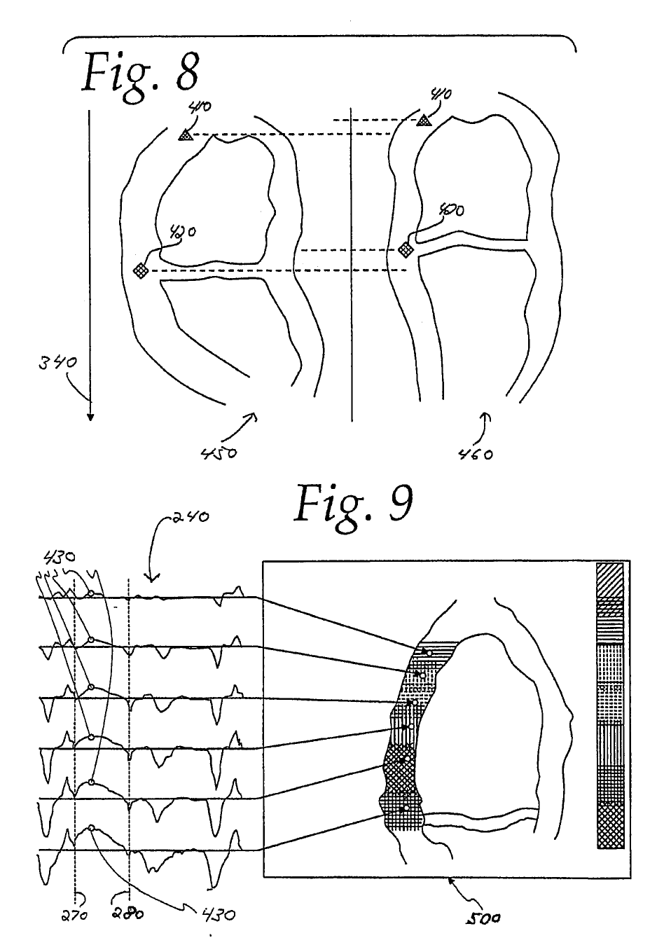

FIG. 8 illustrates how the location of the landmarks (identified by the indicia 410 and 420) may move from end diastole 450 to end systole 460 of the cardiac cycle during live imaging. The motion may be viewed by the operator when the tracking and indicia labeling techniques described above are employed.

Clinical trials may be performed so that locations (depths) of the anatomical landmarks may be anticipated and may be preset in the ultrasound machine. Algorithms and functions for locating the landmarks may be implemented more efficiently by, for example, limiting the part of the motion gradient profile that needs to be searched for peaks.

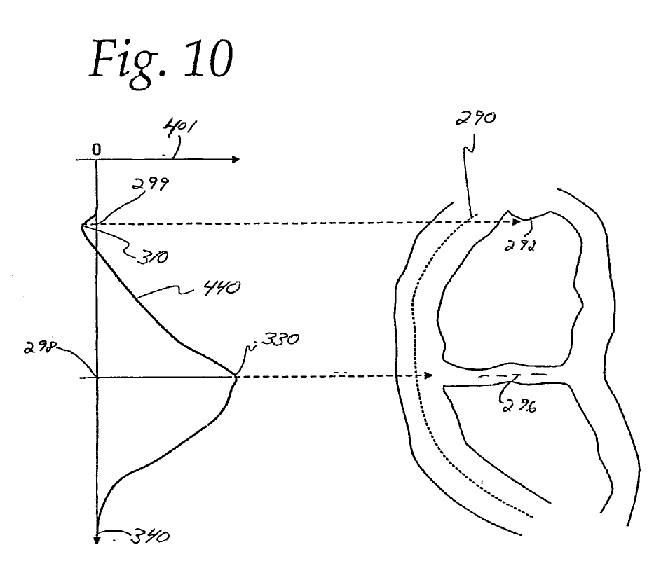

Referring to FIGS. 9 and 10, as one alternative embodiment of the present invention, the estimated tissue velocity values for each designated, tracked anatomical point in the myocardial segment may be peak-detected over the time period Tₛₜₐᵣₜ 270 to T_{end} 280 to construct a velocity gradient profile 440 of peak velocity values 401 instead of integrating the velocity values over time. The peak-detection techniques described above may then be applied to the velocity gradient profile to locate the anatomical landmarks in the same manner previously described. FIGS. 9 and 10 illustrate using peak-detected tissue velocity profiles 240 to generate the peak parameter values 430. Instead of integrating over the time period, the velocity profiles are peak-detected. The resultant velocity gradient profile 440 is constructed over depth 340 from the peak values 430 as shown in FIG. 10. However, construction of the motion gradient profile 320, by integrating the velocities, reduces the noise content in the profile 320 and provides a more robust source for localization of peak values in the gradient profile.

As a further alternative embodiment of the present invention, tissue strain rate values may be generated by Doppler processor 40 and used to generate a strain rate gradient profile for tracked anatomical points within a myocardial segment. Since strain rate is the spatial derivative of velocity, the AV-plane may be located by finding a zero crossing of the profile.

In another alternative embodiment of the present invention, since the mitral valve is connected to the ventricle in the AV-plane, AV-plane localization may be inferred if the mitral valves may be localized. The mitral valves have characteristic shape that may be identified with B-mode imaging and are the tissue reflectors having the highest velocities in the heart. Also, color flow, PW-Doppler, and/or CW-Doppler of blood flow may be used to localize the AV-plane due to known flow singularities across the mitral valve at specific time in the cardiac cycle.

In a further alternative embodiment of the present invention, the position information of the tracked anatomical landmarks may be reported out of the ultrasound machine and/or captured in a storage device for later analysis instead of overlaying indicia on the display corresponding to the anatomical landmarks.

As another alternative embodiment of the present invention, data may be collected and processed in a 3-dimensional manner instead of the 2-dimensional manner previously described.

As still a further alternative embodiment of the present invention, the motion gradient profile 320 (or velocity gradient profile 440) may be displayed along the side of the TVI image on the monitor. The operator may then visualize where the AV-plane 296 and apex 292 are located in the image based on the peaks 310 and 330 in the displayed gradient. The operator may then manually designate the landmark locations as points in the image that may then be automatically tracked.

As still yet another alternative embodiment of the present invention, more than one myocardial segment in the image may be designated and processed at the same time.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. In an ultrasound system for generating an image responsive to moving cardiac structure within a subject, an apparatus for locating anatomical landmarks of said moving cardiac structure comprising:
a front-end arranged to transmit ultrasound waves into the moving cardiac structure and to generate received signals in response to ultrasound waves backscattered from the moving cardiac structure over a time period; and
a processor responsive to said received signals to generate a set of analytic parameter values representing movement along a segment of said moving cardiac structure over said time period, wherein said processor generates said set of analytic parameter values for a given sampled anatomical point within the moving cardiac structure by converting a set of estimated values in a motion parameter profile, and said processor analyzing elements of said set of analytic parameter values to automatically extract position information of said anatomical landmarks.

2. The system of claim 1, wherein said processor generates said set of analytic parameter values by computing a series of time integrals (S₁, S₂ . . . , Sₙ) in which
Sᵢ=T*(V₁+Vₛ+ . . . Vᵢ)
where T is the time delay between two consecutive estimated values, and Sᵢ is a longitudinal distance that a sample of the moving cardiac tissue has moved at time segment Tᵢ.

3. The system of claim 2, wherein said processor further comprises a memory, said processor storing said set of analytic parameter values for each designated anatomical point of the moving cardiac structure as a sampled array of motion values.

4. The system of claim 1, wherein said analytic parameter values comprise a velocity value and a motion value, wherein said processor selects said velocity value for a designated anatomical point in the image from a spatial set of estimated tissue velocity values corresponding to a first time, and said processor computes said motion value for the designated anatomical point using said velocity value.

5. The system of claim 4, wherein said processor comprises a memory, said processor storing said velocity value in a tracked velocity parameter profile array, and said processor stores said motion value in said motion parameter profile.

6. The system of claim 1, wherein said processor locates an apex and AV-plane of the moving cardiac structure by peak-detecting a motion gradient profile over a depth.

7. The system of claim 6, wherein said processor determines the AV-plane by detecting a positive peak, and said processor determines the apex by detecting a negative peak.

8. The system of claim 1, wherein said processor automatically labels discrete anatomical points in the image at longitudinal depths of anatomical landmarks with indicia.

9. In an ultrasound machine for generating an image responsive to moving cardiac structure within a subject, a method for locating anatomical landmarks of said moving cardiac structure comprising:
transmitting ultrasound waves into said moving cardiac structure and generating received signals in response to ultrasound waves backscattered from said moving cardiac structure over a time period;
generating a set of analytic parameter values representing movement along a segment of said moving cardiac structure over said time period in response to said received signals by converting a set of estimated values in a motion parameter profile; and
extracting position information of said anatomical landmarks from said set of analytic parameter values by analyzing elements of said set of analytic parameter values.

10. The method of claim 9, wherein said generating comprising computing a series of time integrals (S₁, S₂ . . . , Sₙ) in which
Sᵢ=T*(V₁₊Vₛ+ . . . Vᵢ)
where T is the time delay between two consecutive estimated values, and Sᵢ is a longitudinal distance that a sample of the moving cardiac tissue has moved at time segment Tᵢ.

11. The method of claim 10, further comprising storing said set of analytic parameter values for each designated anatomical point of the moving cardiac structure as a sampled array of motion values.

12. The method of claim 9, further comprising selecting a first portion of said analytic parameter values for a designated anatomical point in the image from a spatial set of estimated tissue values corresponding to a first time, and computing a second portion of said analytic parameter values for the designated anatomical point using said first portion of said analytic parameter value.

13. The method of claim 11, further comprising storing said first portion of said analytic parameter values in a tracked velocity parameter profile array, and storing said second portion of said analytic parameter values in a motion parameter profile.

14. The method of claim 9, further comprising locating an apex and AV-plane of the moving cardiac structure by peak-detecting a motion gradient profile over a depth.

15. The method of claim 14, wherein said locating an apex and AV-plane comprises determining the AV-plane by detecting a positive peak, and determining the apex by detecting a negative peak.

16. The method of claim 9, further comprising automatically labeling discrete anatomical points in the image at longitudinal depths of anatomical landmarks with indicia.

17. In an ultrasound machine for generating an image responsive to moving cardiac structure within a subject, a method for locating anatomical landmarks of said moving cardiac structure comprising:
generating a timing event source to generate a cardiac timing event signal that represents a cardiac waveform of the subject;
inputting the timing event signal into the ultrasound machine;
transmitting ultrasound waves into said moving cardiac structure and generating received signals in response to ultrasound waves backscattered from said moving cardiac structure over a time period;
designating anatomical points within the moving cardiac structure;
converting a set of estimated tissue velocity values into a motion parameter profile;
creating a tracked velocity parameter profile for at least one of the anatomical points through said converting;
estimating changes in spatial locations of the anatomical points;
extracting velocity estimates based on changes in the spatial locations of the anatomical points;
producing a tracked velocity parameter profile for the at least one of the anatomical points in the image as a function of time for a complete cardiac cycle;
storing the tracked velocity parameter profile as a sampled array of tissue velocity values;
automatically labeling discrete anatomical points corresponding to anatomical landmarks in the image with indicia; and
continuously updating and displaying positions of the indicia to follow movements of the anatomical points.
